(19) United States
(12) Patent Application Publication
(10) Pub. No.: US 2025/0387548 A1
(43) Pub. Date: Dec. 25, 2025

(54) DYNAMIC MEASUREMENT OF CONSTITUENT CONCENTRATION IN AQUEOUS SOLUTIONS

(71) Applicant: Fresenius Medical Care Holdings, Inc., Waltham, MA (US)

(72) Inventors: Perry N. Law, Kaysville, UT (US); Charles Wade Dummer, Waltham, MA (US); Christina Lowry, Waltham, MA (US); Louis LeeGrande Barrett, West Point, UT (US)

(73) Assignee: Fresenius Medical Care Holdings, Inc., Waltham, MA (US)

(21) Appl. No.: 19/248,283

(22) Filed: Jun. 24, 2025

Related U.S. Application Data
(60) Provisional application No. 63/663,646, filed on Jun. 24, 2024.

(51) Int. Cl. A61M 1/16; G01N 21/31; G01N 33/49

(52) U.S. Cl. CPC A61M 1/1603; G01N 21/314; G01N 33/49; A61M 2205/3313; G01N 2021/3148; G01N 2021/3155

(57) ABSTRACT

Constituents of a liquid, such as blood, may exhibit optical signatures that allow for real-time, dynamic concentration detection. For example, concentrations of substances in aqueous solutions, including calcium, potassium, sodium, bicarbonate, chloride, magnesium, phosphate, lactate, acetate, glucose, creatinine, urea, and/or hydrogen peroxide, may be monitored during dialysis or other treatments. This is achieved by measuring real-time changes in light intensity at wavelengths corresponding to the constituents' optical signatures. Concentration may be determined using a predetermined relationship between light intensity and concentration at isolated wavelengths. Real-time monitoring enables timely treatment adjustments, improving patient outcomes.

### CROSS-REFERENCE TO RELATED APPLICATIONS

This application claims benefit to U.S. Provisional Patent Application No. 63/663,646, filed on Jun. 24, 2024, which is hereby incorporated by reference herein.

### FIELD

The present disclosure relates to systems and methods for detecting solution constituents based on optical techniques.

### BACKGROUND

Vascular calcification is the leading cause of mortality in patients with chronic kidney disease. Although there are many precipitating causes for death in this population, positive calcium balance during hemodialysis is one of the contributing events. Similarly, metabolic bone disease is a source of significant morbidity in chronic kidney disease. Negative calcium balance, or removing too much calcium during hemodialysis, contributes to this problem.

Hemodialysis has been standardized and the overwhelming majority of patients are treated with two calcium dialysate baths, 2.5 mEq/L and 3.0 mEq/L. Current practice exposes many patients to either substantial negative or positive calcium balance during hemodialysis. Excess calcium administered during hemodialysis can be at least partially deposited in arteries, contributing to vascular disease. Too much calcium removed from the blood can promote bone loss and potentially contribute to secondary hyperparathyroidism. This is further exacerbated by situations which affect the dialysis calcium gradient such as hyperphosphatemia, calcimimetics administration, and the use of calcium-based phosphorus binders. Similarly, imbalances in potassium, sodium and other blood constituents associated with hemodialysis treatment can have serious consequences for the patient.

### SUMMARY

A first aspect of the present disclosure provides a system for dynamically monitoring constituent concentration changes during dialysis. The system includes a fluid chamber configured to allow a fluid to flow through. The fluid includes a plurality of constituents, and the fluid is extracorporeal blood or dialysate effluent. The system also includes one or more optical sources configured to emit light through the fluid, the light including a plurality of wavelengths corresponding to one or more target constituents and one or more competing constituents in the fluid, one or more optical detectors configured to receive light that has passed through the fluid, the received light corresponding to the plurality of wavelengths, and one or more processors. The one or more processors are configured to: obtain, based on the light received by the one or more optical detectors, intensity information of the light at the plurality of wavelengths, and determine, in real-time, based on the intensity information of the light at the plurality of wavelengths, a change of a concentration of at least one target constituent of the one or more target constituents in the fluid using a plurality of coefficients based on at least one optical signature corresponding to the at least one target constituent. The plurality of coefficients provide for isolating the at least one target constituent from competing constituents in the fluid. Each of the plurality of coefficients corresponds to a respective intensity information for a respective wavelength.

According to an embodiment of the first aspect, the one or more processors are further configured to: perform a response operation based on the determined concentration change of the at least one target constituent. The responsive operation includes adjustment of a treatment parameter or adjustment of a dialysate mixture.

According to an embodiment of the first aspect, the plurality of wavelengths include at least three wavelengths.

According to an embodiment of the first aspect, the one or more processors are further configured to: obtain optical signatures corresponding to the one or more target constituents and one or more competing constituents, each optical signature indicating a set of wavelengths and relationships between the set of wavelengths corresponding to a respective constituent among the one or more target constituents and one or more competing constituents, and determine, for a target constituent or a competing constituent among the one or more target constituents and one or more competing constituents, a set of ratiometric coefficients corresponding to the plurality of wavelengths based on the corresponding optical signature. Determining the change of the concentration of the at least one target constituent is based on at least one set of ratiometric coefficients for the at least one target constituent. The at least one set of ratiometric coefficients for the at least one target constituent is comprised in the plurality of coefficients.

According to an embodiment of the first aspect, the one or more processors are further configured to: determine, based on at least one set of ratiometric coefficients for the at least one competing constituent, a contribution from the at least one competing constituent to the intensity of light, and determine a contribution from the at least one targeting constituent to the intensity of light by subtracting the contribution from the at least one competing constituent. The at least one set of ratiometric coefficients for the at least one competing constituent is comprised in the plurality of coefficients.

According to an embodiment of the first aspect, the one or more processors are further configured to: determine one or more wavelengths of the plurality of wavelengths corresponding to a first target constituent of the one or more target constituents, and determine, based on intensity of light at the one or more wavelengths of the plurality of wavelengths, a change of a concentration of the first target constituent.

According to an embodiment of the first aspect, the one or more processors are further configured to: the one or more optical sources include a first optical source and a second optical source. The first optical source is configured to emit light at a first set of wavelengths and the second optical sources is configured to emit light at as a second set of wavelengths, and wherein the first and second sets of wavelengths comprise different wavelengths.

According to an embodiment of the first aspect, the first set of wavelengths includes one or more wavelengths within the ultraviolet or visible spectrum range, and the second set of wavelengths includes one or more wavelengths within the visible or infrared spectrum range.

According to an embodiment of the first aspect, the one or more optical detectors comprise at least one miniature solid-state spectrophotometer.

According to an embodiment of the first aspect, the one or more optical sources and the one or more optical detectors are configured to continuously detect light from the fluid.

According to an embodiment of the first aspect, the one or more target constituents and the one or more competing constituents in the fluid are instructed by user input.

According to an embodiment of the first aspect, the one or more target constituents include at least one of: calcium, potassium, sodium, chlorine, bromine, bicarbonate, magnesium, phosphate, lactate, acetate, creatinine, glucose, urea, or hydrogen peroxide.

According to an embodiment of the first aspect, a target constituent of the one or more target constituents is potassium.

According to an embodiment of the first aspect, a target constituent of the one or more target constituents is calcium.

According to an embodiment of the first aspect, a target constituent of the one or more target constituents is bicarbonate.

A second aspect of the present disclosure provides a method for dynamically monitoring constituent concentration changes during dialysis. The method includes: emitting light through a fluid that flows through a fluid chamber, the light including a plurality of wavelengths corresponding to one or more target constituents and one or more competing constituents in the fluid. The fluid includes a plurality of constituents, and the fluid is extracorporeal blood or dialysate effluent. The method also includes: receiving light that has passed through the fluid, the received light corresponding to the plurality of wavelengths, obtaining, based on the light received by the one or more optical detectors, intensity information of the light at the plurality of wavelengths, and determining, in real-time, based on the intensity information of the light at the plurality of wavelengths, a change of a concentration of at least one target constituent of the one or more target constituents in the fluid using a plurality of coefficients based on at least one optical signature corresponding to the at least one target constituent. The plurality of coefficients provide for isolating the at least one target constituent from competing constituents in the fluid. Each of the plurality of coefficients corresponds to a respective intensity information for a respective wavelength.

According to an embodiment of the second aspect, the plurality of coefficients include at least one set of ratiometric coefficients corresponding to at least one target constituent of the one or more target constituents and at least one set of ratiometric coefficients corresponding to at least one competing constituent of the one or more competing constituents. The method also includes: determining, based on the at least one set of ratiometric coefficients corresponding to the at least one competing constituent of the one or more competing constituents, a contribution from the at least one competing constituent to the intensity of light, and determine a contribution from at least one targeting constituent of the one or more targeting constituents to the intensity of light by subtracting the contribution from the at least one competing constituent.

According to an embodiment of the second aspect, a target constituent of the one or more target constituents is potassium.

A third aspect of the present disclosure provides non-transitory computer-readable medium, having computer-executable instructions stored thereon for dynamically monitoring constituent concentration changes during dialysis. The computer-executable instructions, when executed, facilitate performance of the following: emitting light through a fluid that flows through a fluid chamber, the light including a plurality of wavelengths corresponding to one or more target constituents and one or more competing constituents in the fluid. The fluid includes a plurality of constituents, and the fluid is extracorporeal blood or dialysate effluent. The computer-executable instructions, when executed, also facilitate performance of the following: receiving light that has passed through the fluid, the received light corresponding to the plurality of wavelengths, obtaining, based on the light received by the one or more optical detectors, intensity information of the light at the plurality of wavelengths, and determining, in real-time, based on the intensity information of the light at the plurality of wavelengths, a change of a concentration of at least one target constituent of the one or more target constituents in the fluid using a plurality of coefficients based on at least one optical signature corresponding to the at least one target constituent. The plurality of coefficients provide for isolating the at least one target constituent from competing constituents in the fluid. Each of the plurality of coefficients corresponds to a respective intensity information for a respective wavelength.

According to an embodiment of the third aspect, the plurality of coefficients include at least one set of ratiometric coefficients corresponding to at least one target constituent of the one or more target constituents and at least one set of ratiometric coefficients corresponding to at least one competing constituent of the one or more competing constituents. The computer-executable instructions, when executed, also facilitate performance of the following: determining, based on the at least one set of ratiometric coefficients corresponding to the at least one competing constituent of the one or more competing constituents, a contribution from the at least one competing constituent to the intensity of light, and determine a contribution from at least one targeting constituent of the one or more targeting constituents to the intensity of light by subtracting the contribution from the at least one competing constituent.

### BRIEF DESCRIPTION OF THE DRAWINGS

Embodiments of the present disclosure will be described in even greater detail below based on the exemplary figures. The present disclosure is not limited to the exemplary embodiments. All features described and/or illustrated herein can be used alone or combined in different combinations in embodiments of the present disclosure. The features and advantages of various embodiments of the present disclosure will become apparent by reading the following detailed description with reference to the attached drawings which illustrate the following:

FIG. 1 illustrates a simplified block diagram depicting a detection system according to one or more examples of the present disclosure.

FIG. 2 is a schematic diagram of an exemplary hemodialysis system having an optical blood monitoring system, according to one or more examples of the present disclosure.

FIG. 3 is a block diagram illustrating a sensor system, according to one or more examples of the present disclosure.

FIG. 4A is an example plot illustrating a group of spectra corresponding to bicarbonate, according to one or more examples of the present disclosure.

FIG. 4B an example plot illustrating a critical wavelength region for bicarbonate, according to one or more examples of the present disclosure.

FIG. 4C is an example parametric plot illustrating correlation between prediction and ground truth, according to one or more examples of the present disclosure.

FIG. 5 is a flowchart of a process for determining and/or monitoring concentration of a constituent according to one or more examples of the present disclosure.

FIG. 6 is a flowchart of a process for determining concentration of a constituent in a sample, according to one or more examples of the present disclosure.

### DETAILED DESCRIPTION

If real-time blood calcium concentration were to be determinable during dialysis treatment, a physician could adjust the dialysis prescription to prevent significant positive or negative calcium balance. This would remove one risk factor for vascular calcification and metabolic bone disease. Similarly, if other constituents of aqueous solutions (including but not limited to potassium, sodium, bicarbonate, chloride, magnesium, phosphate, lactate, acetate, glucose, creatinine, urea, and/or hydrogen peroxide concentrations in blood, dialysate and/or dialysate effluent) were to be determinable in real-time during dialysis treatments or in other contexts, other treatment adjustments could be performed in real-time to provide for better patient outcomes.

Exemplary embodiments of the present disclosure provide devices, systems and methods for rapid and accurate measurement of various constituents of aqueous solutions based on their distinct optical signatures, thereby allowing monitoring of dynamic and/or real-time changes in the concentration of those constituents.

Constituents of an aqueous solution sample, such as extracorporeal blood or spent dialysate (effluent), may have unique optical signatures. For a specific constituent, the corresponding optical signature is indicated by absorption characteristics at wavelengths within a spectrum. At each absorption wavelength, the observed intensity varies predictably with the concentration of a specific constituent. The observed intensity changes for different wavelengths exhibit varying rates of absorption in response to changes in concentration. A set of coefficients corresponding to the different wavelengths for each respective constituent is determined based on these absorption-rate relationships. In some embodiments, the absorption relationships among various wavelengths for a specific constituent is established using Partial Least Squares Regression (PLSR). As such, concentration changes of constituents in a sample (e.g., blood) may be measured by monitoring intensity changes at specific wavelengths based on the corresponding optical signatures of the constituents. In some examples, some wavelengths indicated by the optical signature may be utilized to monitor the corresponding constituent, while some wavelengths may be used to determine the influence of other constituents.

In some instances, optical signatures may be determined based on absorption spectra of specific constituents in a controlled environment (e.g., using specifically formulated water-based solutions). An optical system utilizing one or more light sources and detectors may be used for measuring the optical signatures. Multiple detectors may be utilized to obtain spectra at different wavelength ranges. Broadband light sources may be used to cover a wide wavelength range, for example from ultraviolet (UV) to infrared (IR) regions. Narrowband light sources, such as lasers, may be utilized to make finer wavelength resolution measurements. Using broadband light sources may increase efficiency, while using narrowband light sources may improve accuracy and sensitivity. In some variations, a detection system may utilize both broadband and narrowband light sources to perform spectrum measurement.

By knowing the optical signatures, an optical system can be employed to monitor the concentration of specific constituents in a sample (including, for example, continuous monitoring, periodic monitoring, and/or on-demand monitoring). For example, the optical system may incorporate one or more light sources and detectors operating at precise wavelengths designed for particular constituents, such as calcium and potassium. To this end, the optical system may be tailored to specifically track and monitor these constituents.

In some embodiments, an optical system may be utilized to monitor a group of constituents in an aqueous solution, consisting of one or more target constituents and competing constituents. The target constituents and competing constituents exhibit both overlapping and distinct wavelengths in the spectrum. By utilizing selected wavelengths, the optical system essentially isolates the changes corresponding to the target constituents. The optical system may be configured to monitor constituent concentrations dynamically and in real time.

In an exemplary embodiment involving dialysis treatment systems, monitoring constituents such as bicarbonate and calcium during dialysis offers several important benefits for patient care. Real-time bicarbonate measurement enables personalized adjustment of dialysate bicarbonate levels, which is advantageous for maintaining proper acid-base balance and addressing metabolic acidosis commonly seen in patients with kidney failure. Continuous calcium monitoring supports the accurate management of calcium mass balance, helping to prevent conditions like hypercalcemia and vascular calcification. It also allows for timely adjustments to dialysate calcium concentrations and calcium-based medications, ensuring patients maintain stable and safe calcium levels. Overall, monitoring these constituents, as well as other suitable constituents, enhances treatment precision, supports better clinical outcomes, and reduces the risk of patient complications.

The methods and systems disclosed herein can be utilized in various applications where monitoring the concentration of specific constituents is advantageous. In some embodiments, the present disclosure describes the technology with reference to monitoring specific fluid samples during hemodialysis. However, this example is provided for illustrative purposes only, to facilitate understanding of the principles of the technology, and is not intended to limit the scope of the present disclosure.

A “sample” as contemplated by the disclosure includes liquids, such as blood flowing through an extracorporeal blood circuit during dialysis treatment. It will be noted that the systems/methods of the present disclosure can be applicable to other suitable types of samples, including dialysate, spent dialysate, (effluent), urine, or other medical fluids.

Moreover, the underlying principles are not limited to hemodialysis- or peritoneal dialysis-related applications and may also be extended to other domains, such as monitoring fluid composition in water filtration systems, industrial process control, or environmental testing, where dynamic concentration measurements of fluid constituents are required.

In particular, exemplary aspects of the detection and/or data acquisition systems according to the present disclosure are further elucidated below in connection with exemplary embodiments, as depicted in the figures. The exemplary embodiments illustrate some implementations of the present disclosure and are not intended to limit the scope of the present disclosure.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

Where possible, any terms expressed in the singular form herein are meant to also include the plural form and vice versa, unless explicitly stated otherwise. Also, as used herein, the term “a” and/or “an” shall mean “one or more” even though the phrase “one or more” is also used herein. Furthermore, when it is said herein that something is “based on” something else, it may be based on one or more other things as well. In other words, unless expressly indicated otherwise, as used herein “based on” means “based at least in part on” or “based at least partially on”.

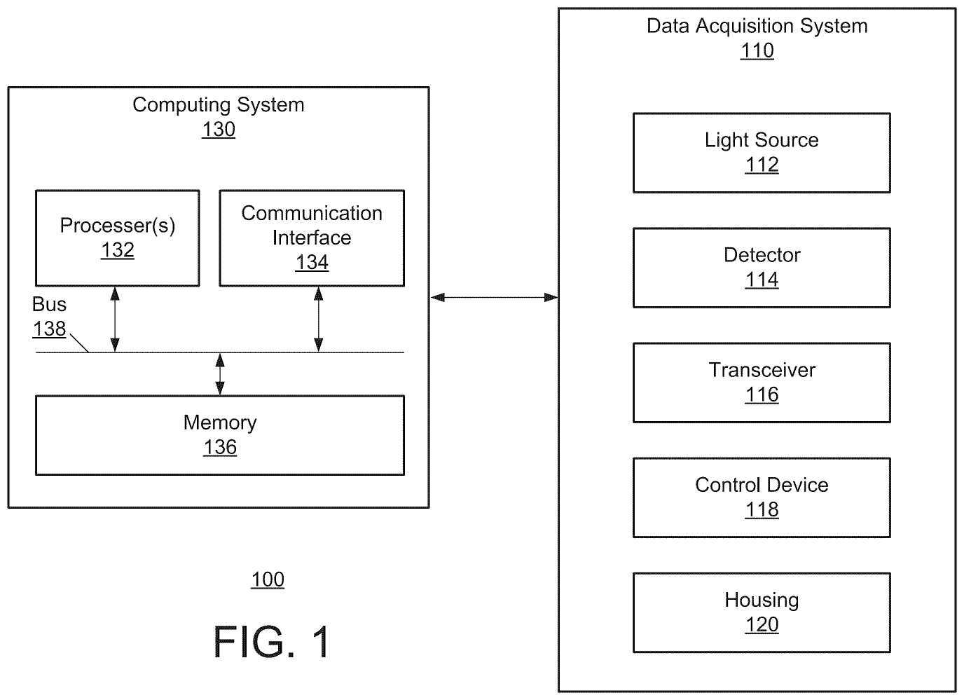

FIG. 1 illustrates a simplified block diagram depicting a detection system 100 according to one or more examples of the present disclosure.

Referring to FIG. 1, the detection system 100 includes a data acquisition system 110 and a computing system 130. The data acquisition system 110 includes various hardware and software components configured to perform data acquisition. The computing system 130 is configured to process data from the data acquisition system 110. In some examples, the computing system 130 may be integrated in or communicatively coupled to the data acquisition system 110. In some instances, the computing system 130 may be communicatively connected to additional sensors (such as temperature sensor, pressure sensor, flow sensor, etc.) to obtain suitable information about the environment in which the detection system 100 is performing tasks.

The data acquisition system 110 includes a light source 112, a detector 114, a transceiver 116, a control device 118, and a housing 120. The housing 120 houses the light source 112, detector 114, transceiver 116, control device 118, and a sample to be measured.

In one or more embodiments, the sample may be a solution having multiple light-attenuating constituents or components. For example, the solution may be blood that has undergone a specific treatment, such as dialysis treatment. Throughout the treatment, the concentration of particular constituents/components within the blood may change dynamically.

The light source 112 is configured to generate incident light, illuminating the sample. Interaction with the sample then induces changes in the spectrum of the incident light. The changes in the spectrum may be attributed to the absorption (or other light attenuation mechanism) of light at particular wavelength(s), which occurs as a result of the concentration of one or more specific constituents/components present in the substance. In some variations, the data acquisition system 110 may include multiple light sources for different regions in the spectrum.

The light source selection encompasses a variety of options, including both broadband and narrowband light sources.

Broadband light sources emit light across a broad range of wavelengths. They provide a continuous spectrum of light, covering a wide range, for example from ultraviolet (UV) to infrared (IR) regions. Broadband light sources, such as tungsten-halogen lamps, deuterium lamps, or xenon arc lamps, may be used in applications like spectroscopy, where a broad range of wavelengths need to be analyzed simultaneously. These sources are particularly useful when studying materials with complex absorption or transmission characteristics across a wide spectral range.

In contrast, narrowband light sources emit light at specific discrete wavelengths or within a narrow range of wavelengths. They produce light with high spectral purity, focusing on specific wavelengths of interest. Examples of narrowband light sources include laser diodes or narrow-bandwidth filters used in conjunction with a white light source. Narrowband light sources are valuable in applications that require precise and selective excitation or measurement of specific absorption or emission features.

The choice between broadband and narrowband light sources depends on the requirements of the measurement or experimental setup. Broadband sources offer a comprehensive view of the entire spectrum, while narrowband sources provide precise control over specific wavelengths for more targeted investigations.

The detector 114 is configured to capture light that has interacted with the sample, allowing for detection of changes in the spectrum resulting from the constituent concentration in the sample. The detector 114 may be specified to detect a specific wavelength range(s). In some variations, the data acquisition system 110 may include multiple detectors 114 configured to detect different wavelength ranges.

Various types of detectors may be used for detecting the spectrum across different wavelength ranges. In one example, semiconductor devices, such as photodiodes, may be utilized for detecting light in the visible and near-infrared (NIR) spectrum. Photodiodes offer high sensitivity, fast response times, and can be used for both continuous and pulsed light measurements. In another example, photomultiplier Tubes (PMTs) may be utilized, which are highly sensitive detectors and may be used in low-light applications across a wide range of wavelengths. In yet another example, spectrometers may be used, which combine a dispersive element (such as a prism or diffraction grating) with a detector to measure the intensity of light at different wavelengths. These detectors can include photodiodes, CCDs, or specialized spectrometer-specific detectors such as linear diode arrays or charge injection devices (CIDs). Spectrometers may be used for precise spectral analysis in a wide range of applications.

The transceiver 116 is configured to receive and transmit data and/or signals from and to the computing system 130, respectively. The data may include measurement data obtained by the detector 114, and optionally configuration information for the data acquisition system 110. For instance, the configuration information may encompass various settings related to the light source 112, such as output power, pulse width, repetition rate, etc. Additionally and/or alternatively, the configuration information may include settings specific to the detector 114, such as the wavelength range, sampling rate, and other suitable parameters. The signals may include control signals that may be used to instruct the control device 118 to control the operation of the light source 112 and/or detector 114 in the data acquisition system 110. For instance, the data acquisition system 110 may receive control signals to start/terminate the data acquisition process, or switch between operation modes (e.g., to detect different wavelength ranges). The data acquisition system 110 may receive the control signals from the computing system 130 or other suitable control devices (e.g., a remote controller). Additionally and/or alternatively, the transceiver 116 may transmit signals indicating status of the data acquisition system 110. For instance, the data acquisition system 110 may include additional sensors to monitor the status of the devices therein. In more detail, the control device 118 may collect signals from the sensors to determine if any of the devices in the data acquisition system 110 is malfunctioning. In the event of an abnormal condition the control device 118 may signal the event via the transceiver 116.

The control device 118 is not constrained to any particular hardware, and the control device's configuration may be implemented by any kind of programming (e.g., embedded Linux) or hardware design—or a combination of both. For instance, the control device 118 may be formed by a single processor, such as a general-purpose processor with the corresponding software implementing the described control operations. On the other hand, the control device 118 may be implemented by a specialized hardware, such as an ASIC (Application-Specific Integrated Circuit), an FPGA (Field-Programmable Gate Array), a DSP (Digital Signal Processor), a GPU (graphics processing unit), an NVIDIA Jetson Device, a hardware accelerator, a processor operating TENSORFLOW, TENSORFLOW LITE, PYTORCH, and/or other ML software, and/or other devices. In some instances, the control device 118 may be an edge computing hardware that is on and/or included within the data acquisition system 110.

Referring back to FIG. 1, the computing system 130 may be a part of or an extension of the control device 118 of the data acquisition system 110. The computing system 130 includes one or more processors 132, a communication interface 134, and a memory 136, which are communicatively coupled to a bus 138. Data can be transmitted between the one or more processors 132, the communication interface 134, and the memory 136 via a bus 138.

The one or more processors 132 are configured to perform operations in accordance with the instructions stored in the memory 136. The processor(s) 132 may be any appropriate type of general-purpose or special-purpose microprocessor (e.g., a CPU or GPU, respectively), digital signal processor, microcontroller, or the like.

The memory 136 is configured to store computer-readable instructions that, when executed by the processor(s) 132, can cause the processor(s) 132 to perform various operations disclosed herein. The memory 136 may be any non-transitory type of mass storage, such as volatile or non-volatile, magnetic, semiconductor-based, tape-based, optical, removable, non-removable, or other type of storage device or tangible computer-readable medium including, but not limited to, a read-only memory (“ROM”), a flash memory, a dynamic random-access memory (“RAM”), and/or a static RAM.

The communication interface 134 is configured to communicate information between the computing system 130 and the data acquisition system 110 as show in FIG. 1. As one example, the communication interface 134 may include an integrated services digital network (“ISDN”) card, a cable modem, a satellite modem, or a modem to provide a data communication connection. As another example, the communication interface 134 includes a local area network (“LAN”) card to provide a data communication connection to a compatible LAN. As a further example, the communication interface 134 may include a high-speed network adapter such as a fiber optic network adaptor, 10G Ethernet adaptor, or the like. Wireless links can also be implemented by the communication interface 134. In such an implementation, the communication interface 134 can send and receive electrical, electromagnetic or optical signals that carry digital data streams representing various types of information via a network. The network can typically include a cellular communication network, a Wireless Local Area Network (“WLAN”), a Wide Area Network (“WAN”), or the like. In some variations, the communication interface 134 may include various I/O devices such as a keyboard, a mouse, a touchpad, a touch screen, a microphone, a camera, a biosensor, etc.

In some instances, the computing system 130 may be implemented using one or more computing platforms, devices, servers, and/or apparatuses. In other instances, the computing system 130 may be implemented as engines, software functions, and/or applications. In other words, the functionalities of the computing system 130 may be implemented as software instructions stored in storage (e.g., memory) and executed by one or more processors.

It will be appreciated that the exemplary detection system 100 depicted in FIG. 1 is merely an example, and that the principles discussed herein may also be applicable to other situations—for example, other types of data acquisition systems 110.

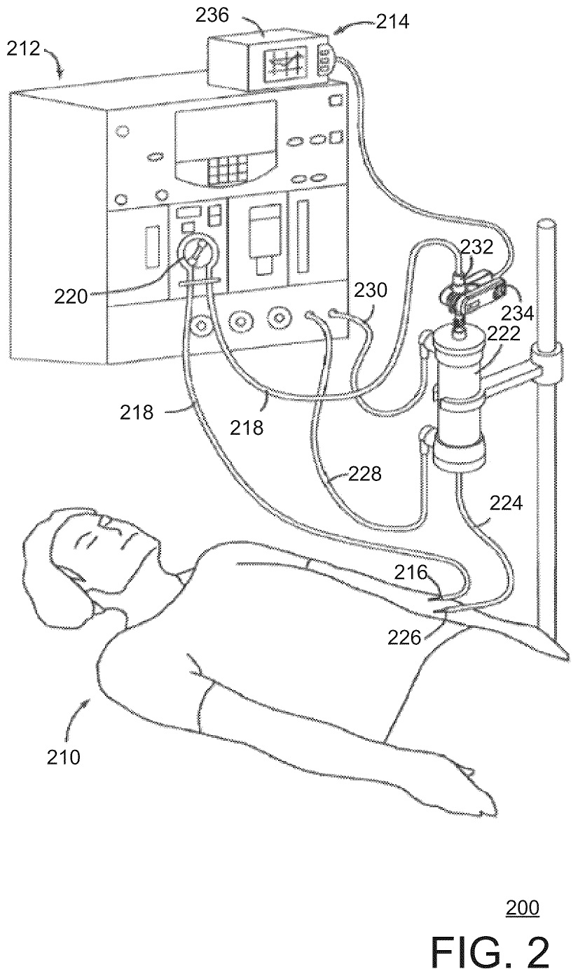

FIG. 2 is a schematic diagram of an exemplary hemodialysis system having an optical blood monitoring system, according to one or more examples of the present disclosure. FIG. 2 depicts a patient 210 undergoing hemodialysis treatment using a hemodialysis machine 212. The hemodialysis system further includes an optical blood monitoring system 214. The optical blood monitoring system 214 is an example of a detection system 100 or data acquisition system 110 shown in FIG. 1.

An input needle or catheter 216 is inserted into an access site of the patient 210, such as in the arm, and is connected to extracorporeal tubing 218 that leads to a peristaltic pump 220 and to a dialyzer 222 (or other blood filter). The dialyzer 222 removes toxins and excess fluid from the patient's blood. The dialyzed blood is returned from the dialyzer 222 through extracorporeal tubing 224 and return needle or catheter 226. Often, the extracorporeal blood flow may additionally receive a heparin drip to prevent clotting. The excess fluids and toxins are removed using clean dialysate liquid which is supplied to the dialyzer 222 via tube 228, and waste liquid is removed for disposal via tube 230. A typical hemodialysis treatment session takes about three to five hours in the United States.

The optical blood monitoring system 214 includes a display device 236 and a sensor device 234. The sensor device 234 may, for example, be a sensor clip assembly that is clipped to a blood chamber 232, wherein the blood chamber 232 is disposed in the extracorporeal blood circuit. A controller of the optical blood monitoring system 214 may be implemented in the display device 236 or in the sensor device 234, or both the display device 236 and the sensor device 234 may include a respective controller for carrying out respective operations associated with the optical blood monitoring system.

The blood chamber 232 may be disposed in line with the extracorporeal tubing 218 upstream of the dialyzer 222. Blood from the peristaltic pump 220 flows through the tubing 218 into the blood chamber 232. The sensor device 234 includes emitters that emit light at certain wavelengths and detectors for receiving the emitted light after it has passed through the blood chamber 232. The blood chamber 232 includes lenses or viewing windows that allows the light to pass through the blood chamber 232 and the blood flowing therein. Alternatively, the blood chamber 232 may be disposed in line with the extracorporeal tubing 218 downstream of the dialyzer 222 or both upstream and downstream of the dialyzer 222 (not shown), or in the dialysate line (not shown). Provision of blood chambers 232 with associated sensor devices 234 both upstream and downstream of the dialyzer 222 may facilitate real-time determination of concentration changes in particular blood constituents associated with the hemodialysis process.

A controller of the optical blood monitoring system 214 uses the light intensities measured by the detectors to determine the concentration or concentration change of a particular constituent(s) (e.g., calcium) based on the corresponding optical signature(s).

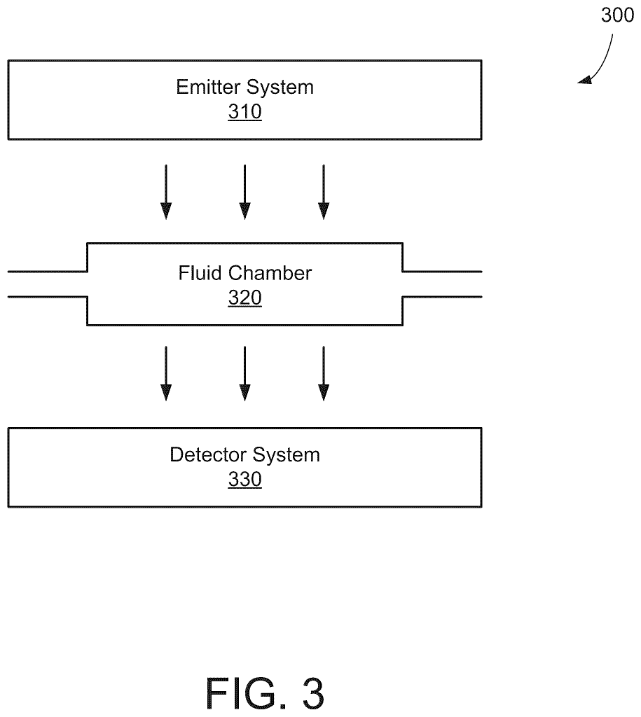

FIG. 3 is a block diagram illustrating a sensor system 300, according to one or more examples of the present disclosure. The sensor system 300 may be implemented in the sensor device 234 in FIG. 2 to facilitate dynamic monitoring of constituent concentration through measurements performed on optical signals. However, it will be noted that other sensor devices that implement the sensor system 300 can also be utilized to perform the methods disclosed herein.

As illustrated in FIG. 3, the sensor system 300 includes an emitter system 310, a fluid chamber 320, and a detector system 330. The fluid chamber 320 is configured to allow a fluid sample to pass through. For example, the fluid chamber 320 may be embodied as the blood chamber 232 in the sensor device 234, through which the patient's blood flows. The fluid chamber 320 includes one or more optically transparent windows that allow light to be incident on and/or emitted from the fluid sample contained within the chamber. This configuration enables interaction between the light and the fluid sample.

In the context of dialysis treatment, the fluid being evaluated may include blood, effluent (i.e., spent dialysate) and/or clean dialysate. The sensor system 300 (e.g., in the sensor device 234) can be configured to monitor various constituents (such as potassium, sodium, bicarbonate, chloride, magnesium, phosphate, lactate, acetate, glucose, creatinine, urea, and/or hydrogen peroxide) of such fluids in real-time during a treatment. By “real-time”, it is understood that fluid constituents may be monitored and evaluated during an actual patient treatment (e.g., dialysis) or other medical procedure essentially immediately, without requiring the fluid sample to be analyzed separately and/or at later time, such as at an analytical laboratory.

The emitter system 310 and detector system 330 within the sensor system 300 operate at one or more wavelengths selected based on the requirements of specific applications.

In at least one embodiment, the emitter system 310 includes an emitter subassembly with a number of light emitters at different wavelengths. For example, to monitor calcium, the emitter subassembly may include two light emitters, one emitting ultra violet (UV) light radiation at one or more first wavelengths (λ1) of about 205 nm, 210 nm, and/or 230 nm and another emitting infrared (IR) light radiation at one or more second wavelengths (λ2) of about 950 nm and/or 1150 nm. Additionally and/or alternatively, the emitter subassembly may include light emitters emitting light radiation at multiple UV wavelengths, IR wavelengths, visible (VIS) wavelengths, or any combination thereof. Other wavelengths could be substituted or added to measure additional blood constituents or properties of other fluids.

Accordingly, the detector system 330 includes a photodetector subassembly with one or more photodetectors. The one or more photodetectors operate within one or more spectral ranges that cover the emitted wavelengths. In at least one embodiment, the one or more photodetectors includes a photodetector to detect the UV region, a photodetector to detect the VIS region, and/or a photodetector to detect the IR region.

In at least one embodiment, the emitter system 310 and the detector system 330 within the sensor system 300 can adopt miniature solid-state components to achieve a compact design. For example, the emitter system may utilize light-emitting diodes or laser diodes, and the photodetectors may implement photodiode detectors.

In at least one embodiment, the detection system 100 utilizes the sensor system 300 to obtain real-time, dynamic measurement results by monitoring fluid flowing through a predefined chamber (e.g., the fluid chamber 320), which lies in the optical path between the emitter system 310 and the detector system 330. The detection system 100 can implement the methods disclosed herein to dynamically determine the concentration of one or more constituents/components in the fluid. The following description presents these methods in the context of concentration measurements for dialysis-related applications. However, it should be noted that the principles of the present disclosure are equally applicable to concentration measurements in other suitable contexts.

When applied to dialysis treatment, the detection system 100 utilizes optical signatures to measure particular constituents/components dynamically as blood (or other relevant fluid) flows in the bloodline or other fluid line during a treatment.

As established above, optical signatures are characterized by specific (or key) wavelengths or peaks that can be used to detect the presence and concentration of a single constituent/component or group of constituents/components within the measured sample, such as blood. These wavelengths or peaks represent key points of interest where significant absorption, reflection, or scattering occurs, providing valuable information about the composition and/or properties of the sample under investigation.

A wide range of constituents, such as calcium and potassium, may be monitored or investigated by implementing the systems and/or methods disclosed herein. A sample, such as blood or dialysate is comprised of a plurality of constituents, consisting of both target and potentially optically competing or interfering constituents. For example, calcium and potassium may be target constituents. Calcium and potassium exist as positive ions in water and the body. They are balanced by various negatively charged ions. A positive ion is referred to as a cation and a negative ion is referred to as an anion. Other anions and/or cations, aside from calcium and potassium, may act as competing constituents.

In at least one embodiment, an optical signature corresponding to a specific constituent is represented by a set of wavelengths, with a set of ratiometric coefficients across those wavelengths that characterize the correlation among them—for example, indicating their relative intensities within the spectrum. In at least one embodiment, a subset of wavelengths from the optical signature is used to obtain concentration information. For example, the sensor system 300 may be configured with a predefined number of detection wavelengths (e.g., five wavelengths). Accordingly, the detection system 100 may be designed to tailor the optical signatures of target and competing constituents to sets of ratiometric coefficients corresponding only to the detected wavelengths. By analyzing the contributions associated with these sets of ratiometric coefficients, the detection system 100 determines the concentrations of one or more target constituents, while isolating or distinguishing them from competing constituents.

In some examples, one or more detected wavelengths may be used to positively correlate the concentration of the constituent of interest with the amplitude of the detected wavelengths. Additionally and/or alternatively, one or more detected wavelengths may be used to subtract the influence from a competing constituent(s) in certain wavelength regions. Table 1 below provides an exemplary list of ions and other chemical species that may be monitored according to the present disclosure.

TABLE 1

| Constituents | Found in chemical form |
|---|---|
| Calcium | CaCl₂, CaBr₂ |
| Potassium | KCl, KBicarbonate |
| Sodium | NaBr, NaBicarbonate, NaLactate, |
| | NaPhosphate, NaAcetate |
| Chlorine | CaCl₂, KCl, MgCl₂ |
| Bromine | CaBr₂, NaBr |
| Bicarbonate | NaBicarbonate, Potassium |
| | Bicarbonate |
| Magnesium | MgCl₂ |
| Phosphate | NaPhosphate |
| Lactate | NaLactate |
| Acetate | NaAcetate |
| Creatinine | Creatinine |
| Glucose | Glucose |
| Urea | Urea |
| Hydrogen Peroxide | Hydrogen Peroxide |

For example, in blood or dialysate samples, calcium and potassium may be target constituents of interest. Sodium and chloride are among the top electrolytes present in spent dialysate solution or in blood, making them biologically relevant. Sodium and chloride can act as possible competing constituents to the target constituents. For example, sodium is electronically similar to potassium, while chloride may compete with other negative ions (e.g., bromine or bicarbonate) that pair with the target ions of calcium or potassium. Magnesium (electronically similar to calcium), phosphate, lactate, acetate, creatinine, glucose and urea (among other chemical species) are biologically relevant. Lactate, acetate and glucose may be present in dialysate (e.g., from peritoneal dialysis or hemodialysis). Creatinine may be used as a reference for many standard tests. Additionally, hydrogen peroxide is a commonly used sanitizing and cleaning agent which exhibits absorbance peaks in the visible region of the spectrum.

In at least one embodiment, the optical signatures corresponding to various constituents are determined in a controlled environment. For example, aqueous solution samples containing one or more selected constituents at predetermined concentrations may be prepared and measured using the detection system 100 disclosed herein. In some examples, the data acquisition system 110 is configured to obtain measurement data from the controlled samples, and the computing system 130 is configured to process the data obtained via the data acquisition system 110.

In at least one embodiment, the data acquisition system 110 transmits the collected data to the computing system 130 that is communicatively coupled to the data acquisition system 110. The computing system 130 implements custom software that is designed and coded to distinguish between light opacity changes and changes due to unique constituent light absorption and scattering.

In at least one embodiment, baseline data is obtained using the data acquisition system 110 to account for and remove the impact of background noise. For example, the baseline data may correspond to measurements of deionized water used as a base medium to dissolve the constituents/components being tested.

In at least one embodiment, the data processing for establishing an optical signature for a specific constituent involves multiple stages. For example, at a first stage, the data processing involves using a custom-built spectrometer software to obtain suitable spectra (e.g., absorption spectra) corresponding to various constituents/components in the controlled samples (e.g., aqueous solutions with varying but known concentrations of specific constituents/components). In some instances, this stage may be performed during data collection. In some embodiments, spectra from the deionized water baseline and spectra from the test sample are used to find an absorption value at each wavelength of the sample using the absorption

$${A}_{λ} = {log}_{10} ( {I}_{0 λ} / {I}_{λ} ) , \qquad ( Eq . 1 )$$

where A denotes absorption, I₀ is the baseline spectra of deionized water, I is the spectra of the sample, and λ denotes wavelength.

In the next stage, the absorption values are paired with concentration values and separated into groups based on common cations, anions, or other chemical similarity among the samples.

For example, a plurality of controlled aqueous solution samples may be prepared, with each sample containing a specific chemical species. Table 2 provides groupings of seven chemical species corresponding to seven exemplary constituents.

As shown in Table 2, certain constituents may be present in the form of multiple chemical species. For example, calcium is found as part of calcium chloride and also as part of calcium bromide. Additionally, bicarbonate is a common component in both the sodium bicarbonate and potassium bicarbonate form. Accordingly, the absorption spectra of multiple aqueous solutions, such as the sodium bicarbonate and potassium bicarbonate samples, each containing known but varying concentrations of the two bicarbonate-containing salts, can be analyzed to study their individual and combined spectral characteristics. In some cases, an aqueous solution sample may contain only a single constituent/component. For example, a sample may be prepared to contain only glucose, as illustrated in Table 2.

TABLE 2

| Chemical Species | Calcium | Potassium | Sodium | Chloride | Bromide | Bicarbonate | Glucose |
|---|---|---|---|---|---|---|---|
| Calcium Chloride | X | | | X | | | |
| Calcium Bromide | X | | | | X | | |
| Potassium Chloride | | X | | X | | | |
| Potassium Bicarbonate | | X | | | | X | |
| Sodium Bromide | | | X | | X | | |
| Sodium Bicarbonate | | | X | | | X | |
| Glucose | | | | | | | X |

Accordingly, at a first stage, absorption spectra for the seven chemical species are obtained for a plurality of predefined concentrations. In the next stage, the absorption spectra are grouped based on common cations or anions.

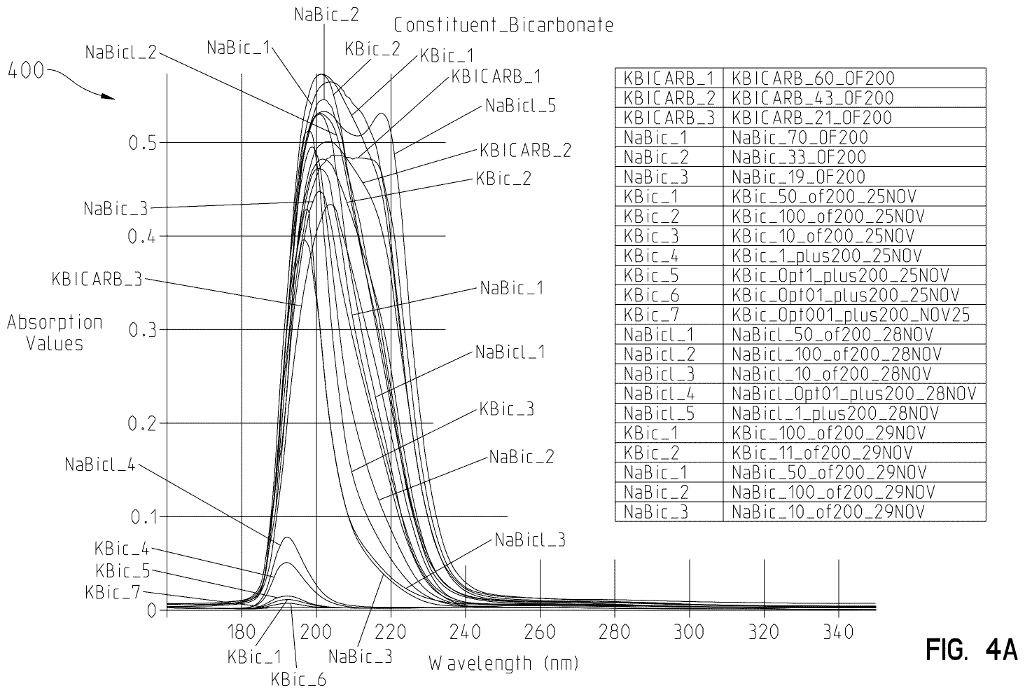

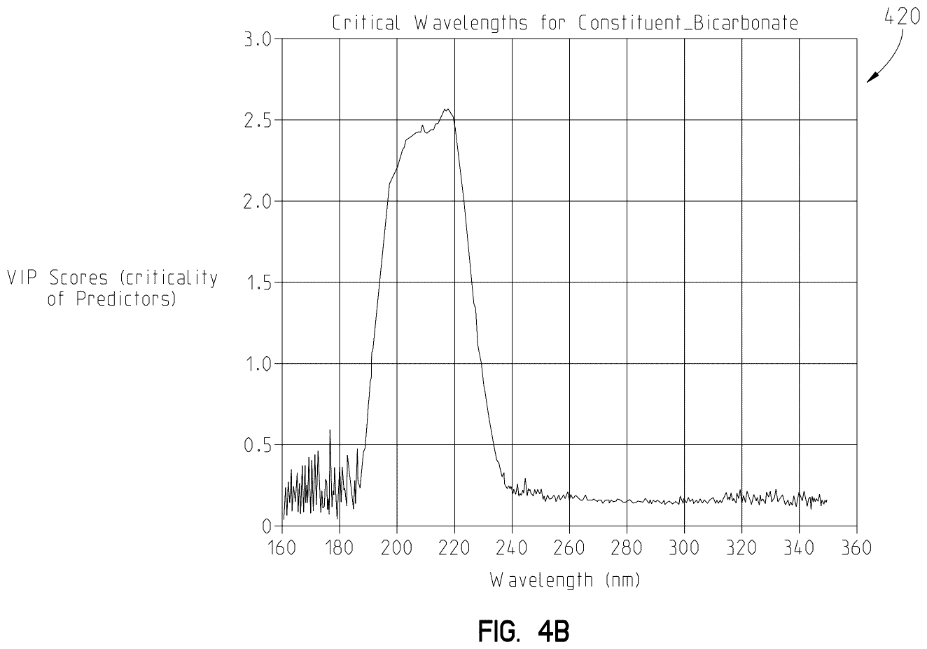

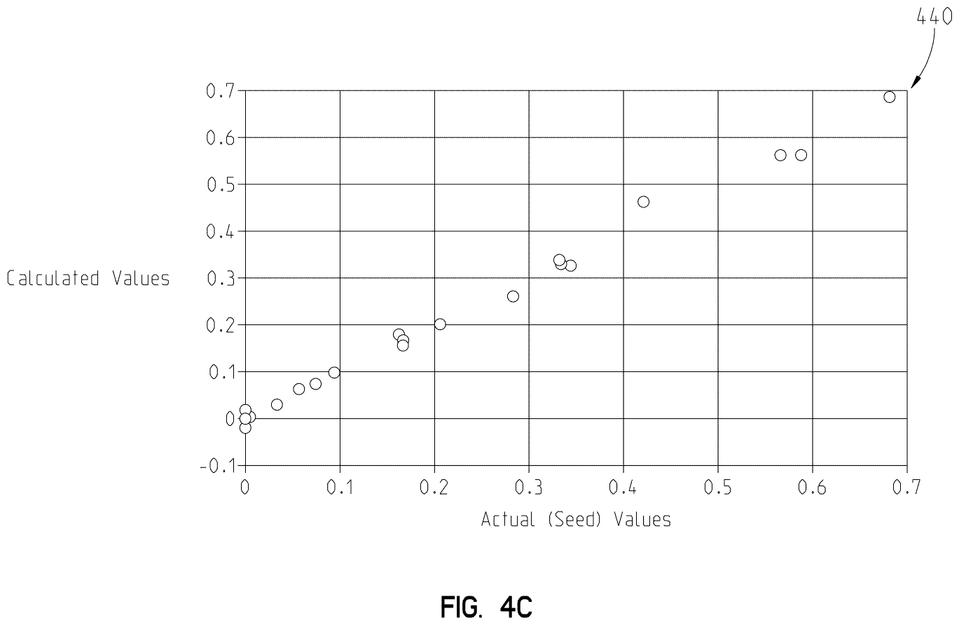

FIG. 4A is an example plot 400 illustrating a group of spectra corresponding to bicarbonate, according to one or more examples of the present disclosure. In FIG. 4A, each curve represents a spectrum obtained from a specific chemical species at a particular concentration. As indicated in the legend, the test involves two chemical species: potassium bicarbonate and sodium bicarbonate, are used for the test. In the illustrated example, the spectra cover only a range of wavelengths within the UV region.

In at least one embodiment, the computing system 130 analyzes the group of spectra in plot 400 to identify one or more wavelengths within the illustrated wavelength region that are prominent in indicating the concentration of bicarbonate in the prepared aqueous sample solutions. In at least one embodiment, the computing system 130 employs suitable multivariate numerical methods, such as Partial Least Squares Regression (PLSR), to identify critical wavelengths or wavelength regions associated with one or more individual constituents.

FIG. 4B is an example plot 420 illustrating a critical wavelength region for bicarbonate, according to one or more examples of the present disclosure. The critical wavelength region is determined based on the group of spectra as illustrated in FIG. 4A. In FIG. 4B, the degree of criticality is represented along the ordinate by a parameter referred to as the VIP Score, with wavelength plotted along the abscissa.

In addition, the computing system 130 determines a set of gain factors, such as weights or ratios, that represent specific relationships among various critical wavelengths (or wavelength regions) associated with a given constituent. For example, the set of gain factors may reflect the relative intensities at different critical wavelengths (or wavelength regions) in the absorption spectra as the concentration of the respective constituent varies. In at least one embodiment, a set of gain factors corresponding to a given constituent is referred to as a set of ratiometric coefficients. In at least one embodiment, an optical signature is represented by a set of critical wavelengths (or wavelength regions), along with a set of ratiometric coefficients.

In an example, the computing system 130 utilizes the established optical signature for bicarbonate to predict concentration in the prepared sample solutions. FIG. 4C is an example parametric plot 440 illustrating correlation between prediction and ground truth, according to one or more examples of the present disclosure. In FIG. 4C, predicted values are plotted along the ordinate, and known (seed) values, given in millimoles per liter (mM), are shown along the abscissa, based on analysis using 11 discrete wavelength regions between 180 nm and 330 nm. In this example, the two times the standard deviation (2SD) of the residuals between known and estimated concentrations is approximately 0.014 mM.

In at least one embodiment, the computing system 130 analyzes data collected over a broad spectral range (e.g., including UV, VIS, or IR regions) to determine an optical signature for a target constituent. In some examples, the computing system 130 refines the optical signature through multiple iterations. At each iteration, the computing system 130 may identify a set of critical wavelengths (or wavelength regions) along with a corresponding set of ratiometric coefficients, forming the iteration-specific version of the optical signature. Based on this analysis, the computing system 130 may decide to remove one or more wavelengths (or wavelength regions) from the current signature to generate a candidate optical signature for evaluation in the next iteration. In at least one embodiment, the computing system 130 stores the optical signatures obtain for a plurality of constituents in a database or another suitable form.

Optical signatures of various constituents enable the prediction of concentration changes in samples with unknown concentrations. For example, using the critical wavelengths and the corresponding gain factors (or ratiometric coefficients), concentration of various constituents in the solutions under test can be estimated. In at least one embodiment, the computing system 130 can use a selected subset of wavelengths and their corresponding ratiometric coefficients to determine the concentration of a specific constituent.

In some embodiments, the computing system 130 estimates the concentration of one or more specific constituents based on multivariate computations using ratiometric comparisons. For example, the computing system 130 may obtain absorption values for a plurality of target wavelengths based on obtained spectra data. In some cases, the absorption values for the target wavelengths may include mixed spectral information associated with multiple constituents. To resolve this, the computing system 130 first determines which constituents are likely contributing to the absorption data at those wavelengths. Then, the computing system 130 obtains the corresponding optical signatures, or ratiometric coefficient sets, associated with those constituents. Using these coefficient sets, the computing system 130 analyzes the spectral intensity changes at the target wavelengths to determine the individual contributions of each constituent.

It should be noted that the methods described herein are applicable to any selected optically active constituent (where “optically active” refers to any means of attenuation, such as absorption or scattering), as well as for any combination of constituents, provided that solutions of known concentrations can be prepared and measured for coefficient derivation.

In at least one embodiment, optical signatures for various constituents may be stored in various forms, including, but not limited to, tables, lists, and metadata. The optical signatures may be stored in the memory 136 or another suitable storage medium in the computing system 130, and are accessible by the processor(s) 132. In at least one embodiment, only partial information of the optical signatures is stored, for example, specifically tailored to the detection wavelengths configured in the sensor system 300.

In some embodiments, an optical signature for a specific constituent can span multiple spectral ranges, such as the UV, visible, and/or IR ranges. In real-world samples, such as blood or dialysate, signals from various constituents are typically intermixed. Having the individual optical signatures enables the separation of this mixed spectral information, allowing for the extraction of target constituent data. For example, in the UV range, calcium sensitivity is greatest in the UV region at 205 nm, 210 nm, and 230 nm. These peaks overlap with other constituent peaks. Data near 210 nm has the least amount of overlap and may be a discernable shoulder even in relatively complex solutions. In some cases, data from the 210 nm region could be used in conjunction with data from the 205 nm region or the 230 nm region to isolate signals in calcium. Competition near 205 nm includes magnesium, phosphate, and chlorine as well as partial overlap from glucose and potassium peaks. Competition near 230 nm includes acetate, bromine, and sodium with partial overlap from urea and lactate.

Accordingly, in one embodiment and as an illustrative example, an optical system (e.g., the detection system 100, or the sensor system 300) monitors calcium concentration in a sample, such as blood, by measuring absorbance at several wavelengths around 205 nm in the UV range and/or within the NIR range. Using a proprietary algorithm and derived coefficient set (e.g., the absorption rates) specific to calcium, the optical system calculates the calcium concentration within the complex solution, where a complex solution is one containing multiple optically absorbing or scattering constituents. It will be noted that the wavelengths discussed herein are provided as examples. The principles of the techniques disclosed herein can be applied to any other suitable wavelengths, spectrum regions, and/or target/competing constituents.

With regard to an optical signature for potassium in the UV range, areas of interest for potassium in the UV range also overlap with other species. Potassium has relatively distinct peaks near 210 nm and 215 nm. The peak near 210 nm is at the far right side of peaks for magnesium, phosphate, chlorine, and calcium and has the possibility to be a discernable peak shoulder even in relatively complex solutions. Potassium's peak near 215 nm may be hidden below peaks for bicarbonate, urea, and lactate. In this case, the influence of bicarbonate may be determined based on spectral data in the near-infrared (NIR) range and subsequently subtracted from the UV spectral data to account for its contribution.

With regard to an optical signature for glucose in the UV range, there is a clear and distinct peak near 200 nm, and with regard to an optical signature for creatinine in the UV range, there is a clear and distinct peak near 270 nm.

According to optical signatures in the NIR range, key peaks for calcium are seen near 950 nm and 1150 nm with additional more competitive peaks near 1300 nm and 1650 nm. Peaks can also be seen for bromide and chloride near 950 nm and 1150 nm.

Peaks for potassium are relatively distinct near 1300 nm and near 1650 nm. When both potassium chloride and potassium bicarbonate are present, some overlap with chloride peaks and bicarbonate is expected; however, using bicarbonate's unique peak near 1200 nm offers possibilities to isolate and account for bicarbonate contributions. In order to use the 1300 nm peak for potassium, other peaks may be used to de-commutate bicarbonate, calcium, chlorine, bromide, and sodium. One way to do this is to use the data near 950 nm and 1150 nm to de-commutate calcium, bromine and chlorine while the data near 1200 nm could be used to subtract out bicarbonate.

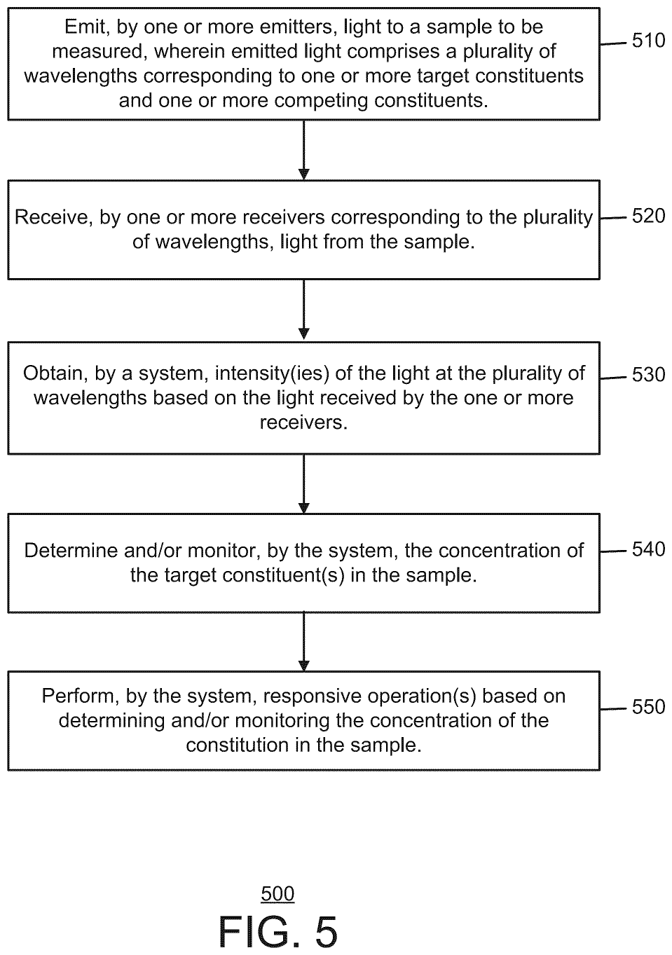

FIG. 5 is a flowchart of a process 500 for detecting and/or monitoring constituent concentration in a fluid passing through a fluid chamber, according to one or more examples of the present disclosure. For example, process 500 may be performed to monitor calcium concentration in an extracorporeal blood circuit of a hemodialysis treatment system. The detection system may be embodied as the detection system 100 shown in FIG. 1. However, it will be recognized that any of the following blocks may be performed in any suitable order and that the process 500 may be performed in any suitable environment and by any suitable data acquisition system and/or computing system.

In this example, the detection system includes one or more emitters (e.g., light sources) configured to emit light at one or more wavelengths, one or more receivers (e.g., detectors) configured to receive light that was emitted from the one or more emitters and that has passed through the sample to be measured, and a computing system to process the data. The computing system includes memory storing a number of predetermined coefficients corresponding to optical signatures corresponding to a number of constituents that are used to estimate constituent concentrations in a test media. The computing system may be, for example, part of or connected to a medical treatment system such as a dialysis machine.

Each optical signature may indicate a specific set of wavelengths exhibiting a predictable (e.g., linear) relationship between the intensity of light at a respective wavelength and the concentration of the constituent. In at least one embodiment, the set of wavelengths corresponds to a set of ratiometric coefficients that indicate the relative relationships among their spectral intensities.

In some examples, the detection system may be customized to suit specific applications. For instance, the emitters and detectors in the detection system may be set to operate at fixed wavelengths, specifically tailored for monitoring calcium and potassium concentrations in blood and/or dialysate liquid during dialysis treatment. In this context, the computing system, whether integrated in or connected to the optical system, may execute a program designed to use optical signatures corresponding to calcium and potassium, or a customized version thereof, for constituent concentration analysis. In some instances, the detection system can be designed as a tunable optical system, allowing it to operate at various wavelengths or wavelength ranges based on specific requirements. This tunable capability offers flexibility and adaptability to address different applications and target various constituents. For instance, the emitters and detectors in the tunable optical system can be adjusted to work at different wavelengths or within specific wavelength ranges, enabling precise detection/monitor of diverse constituents in a sample. To leverage this tunable feature effectively, a dedicated control/computing system may be integrated into or connected with the optical setup. The control/computing system may control and adjust the optical parameters, select appropriate wavelengths, and process the collected data accordingly. By employing suitable algorithms and spectral analysis techniques, the computing system may accurately identify and quantify the concentrations of the targeted constituents based on their corresponding optical signatures.

In some variations, appropriate optical components (e.g., optical filters, gratings, prisms, etc.) may be employed to facilitate the selection of wavelengths in the optical path.

At block 510, the one or more emitters emit light to a sample to be measured. The emitted light comprises a plurality of wavelengths corresponding to one or more target constituents and one or more competing constituents in the sample. The one or more emitters may include one or more broadband light sources or narrowband light sources (such as lasers). In some variations, a tunable laser source may be used as the light emitter. A controller (e.g., the control device 118 shown in FIG. 1) in the detection system may send control signals to the one or more emitters and/or one or more receivers to control the operation status of these components. In one example, the controller may send control signals to a tunable laser to change wavelength of the emitted light. In another example, the controller may send control signals to turn on/off some or all of multiple emitters (e.g., LEDs or lasers) to control the wavelengths included in the emitted light. Accordingly, the controller may switch on/off one or more receivers to ensure effective coverage of the wavelengths included in the emitted light.

At block 520, the one or more receivers receive the light from the sample. The one or more receivers are configured to receive light at the one or more wavelengths, after the light emitted from the one or more emitters has interacted with the sample. The one or more receivers may detect light intensity at one or more wavelength ranges covering the plurality wavelengths emitted by the emitter(s).

In some examples, the one or more receivers may scan/sweep the wavelength range and generate a spectrum at each scan/sweep. The one or more receivers may generate a series of spectra by periodically performing wavelength scans/sweeps. In some instances, the series of spectra may be time-stamped.

At block 530, the computing system obtains intensity(ies) of light at the plurality of wavelengths based on the light received by the one or more receivers. For example, in an implementation, the computing system may first process a series of raw spectra to generate a series of absorption spectra by comparing the series of raw spectra with a baseline (e.g., the spectrum of the light sources, and/or the spectrum of a reference constituent, such as creatinine). Then, the detection system may determine a difference in intensity at a respective wavelength of the optical signature based on the comparison.

In an example implementation, the detection system may retrieve the optical signature of a specific constituent from a memory which stores a plurality of optical signatures corresponding to a plurality of constituents. In an embodiment, the optical signatures stored in memory are reduced to include only the wavelengths detectable by the detection system, along with their corresponding coefficient sets. The detection system may obtain the ratiometric coefficients at the measured wavelengths for the one or more target constituents and/or the one or more competing constituents. Using this information, the detection system may determine the contributions to spectral intensity changes from the target constituent(s) and/or any competing constituent(s).

In some embodiments, the detection system may leverage spectral information at certain measured wavelengths to deconvolute the contributions of competing constituent(s) from those of the target constituent(s) at other wavelengths. For example, the detection system may analyze intensity changes at one or more first wavelengths associated with competing constituents. The detection system may then use these results to mitigate interference from the competing constituents on one or more target constituents at one or more second wavelengths.

At block 540, the detection system determines and/or monitors the concentration of the target constituent(s) in the fluid. The detection system determines the concentration value based on a predictive relationship between the intensity at one or more specific wavelengths and the concentration, for example, as defined by the respective optical signature. For example, the computing system may retrieve the predetermined relationship between the intensity of the one or more wavelengths and the concentration of the constituent from the memory, and then compute the constituent's concentration based on the relationships and the measured intensity values corresponding to the one or more wavelengths corresponding to the optical signature.

In at least one embodiment, the detection system may execute blocks 510-540 to perform the following steps: First, the detection system is configured to measure intensity changes at a plurality of wavelengths, which correspond to one or more target constituents as well as one or more competing constituents. Next, the detection system isolates the intensity changes at one or more first wavelengths from the plurality of wavelengths. These isolated intensity changes represent a composite signal influenced primarily by the optical properties of the competing constituents. Using this information, the detection system can determine and subtract the contributions of the competing constituents to isolate the intensity changes associated with the target constituents. Finally, the detection system determines the concentration changes of the target constituents based on the corrected intensity changes at the relevant wavelengths.

The determined and/or monitored constituent concentration may be displayed on a screen of the computing system, for example, on or near a medical treatment device or at a remote location for remote monitoring and/or control, so that a medical practitioner and/or a patient undergoing treatment can view and/or evaluate the current determined concentration(s) and/or trend(s) corresponding thereto.

In some examples, the detection system may utilize additional parameters to determine the concentration of the constituent. For example, the detection system may utilize additional parameters, such as the volume, flow rate, PH or other suitable parameters of the sample to be measured, to aid in calculating the concentration of constituents, Additionally and/or alternatively, the detection system may apply the additional parameters to determine a confidence level for the measured constituent concentrations.

For example, when the system/method is utilized to measure the calcium mass balance in hemodialysis treatment, the detection system may obtain parameters of dialysis prescription, including treatment time, the blood volume processed, etc. The detection system may calculate calcium balance during the treatment, by applying the optical signature and these additional parameters.

At block 550, the detection system and/or a medical treatment system corresponding thereto may perform responsive operation(s) based on a determination of constituent concentration and/or based on ongoing monitoring of constituent concentration. Responsive operation(s) may include, for example, generating alarms or notifications, as well as modifying or stopping treatments.

In one example, the detection system may implement the process 500 to monitor calcium mass balance during dialysis treatment, and the detection system thus continuously or periodically monitors intensity(ies) at one or more wavelengths corresponding to the optical signature for calcium. Based on the monitored intensity(ies), the detection system may compute the concentrations of calcium dynamically, and when the detection system determines a rate of calcium influx or efflux above a threshold or an absolute concentration above or below a threshold, the detection system may trigger an alarm or other responsive operation(s) (e.g., stopping, slowing down, or speeding up treatment) based thereon. In another example, the detection system may adjust dialysate mixing to control the amount of calcium or sodium in dialysate (which may include generating instructions to clinicians). In other examples, the constituent concentration may be obtained by the detection system to supplement (or replace) lab data to facilitate treatment adjustments, such as with respect to treatment speed or volume of fluid removed, or with respect to patient prescriptions (such as reducing prescribed dosages of calcium-based phosphate binders and vitamin D analogs, which can lead to elevated calcium levels).

In at least one embodiment, real-time calcium monitoring can detect fluctuations and help tailor both dialysate calcium levels and medication adjustments to avoid high calcium levels. Real-time bicarbonate monitoring can also assist with maintaining appropriate levels of bicarbonate during dialysis.

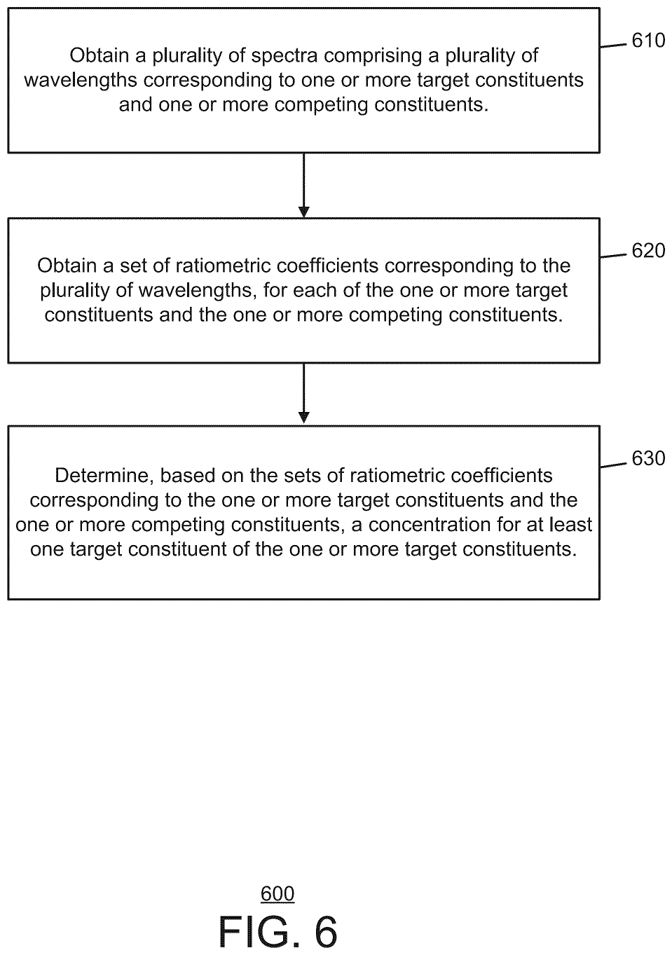

FIG. 6 is a flowchart of a process 600 for determining concentration of a constituent in a sample, according to one or more examples of the present disclosure. In at least one embodiment, process 600 may be performed by the computing system 130 in the detection system 100 shown in FIG. 1. The computing system 130 may receive spectra data from the data acquisition system 110 or another suitable data acquisition system. It will be recognized that any of the following blocks may be performed in any suitable order and that the process 600 may be performed in any suitable environment and by any suitable data acquisition system and/or computing system.

At block 610, the computing system obtains a plurality of spectra including a plurality of wavelengths corresponding to one or more target constituents and one or more competing constituents.

In at least one embodiment, the plurality of wavelengths are contained within one or more wavelength regions that fall within the operation or detection range of the optical components, such as one or more optical emitters and/or one or more optical detectors (or receivers).

In at least one embodiment, the computing system receives instructions indicating the one or more target constituents and/or the one or more competing constituents associated with the plurality of spectra. In some examples, the instructions are provided by user input.

At block 620, the computing system obtains a set of ratiometric coefficients corresponding to the plurality of wavelengths, for each of the one or more target constituents and the one or more competing constituents.

In at least one embodiment, the sets of ratiometric coefficients corresponding to the plurality of wavelengths are derived from a more complete version of the optical signatures associated with the target and competing constituents. For example, the optical signature for constituent 1 may encompass over 20 wavelengths along with their corresponding ratiometric coefficients, whereas in this particular example, only a subset of five wavelengths and their associated coefficient set are used.

In at least one embodiment, the computing system stores only customized versions of the optical signatures, each tailored to the detection capabilities of the optical system with which the computing system is used.

At block 630, the computing system determines, based on the sets of ratiometric coefficients corresponding to the one or more target constituents and the one or more competing constituents, a concentration for at least one target constituent of the one or more target constituents.

To illustrate a simplified example, the spectra obtained from block 610 may include measurements at wavelengths λ₁, λ₂, λ₃, λ₄, and λ₅. C1 and C2 may represent target constituents, and C3 may represent a competing constituent. At block 620, the computing system may determine, based on the ratiometic coefficient sets, a first ratiometic relationship A₁λ₁+B₁λ₂+C₁λ₃+D₁λ₄+E₁λ₅ for C1, a second ratiometic relationship A₂λ₁+B₂λ₂+C₂λ₃+D₂λ₄+E₂λ₅ for C2, and a third ratiometic relationship A₃λ₁+B₃λ₂+C₃λ₃+D₃λ₄+E₃λ₅ for C3. Here, Aₓ through Eₓ represent the ratiometric coefficients associated with the respective wavelengths λ₁ through λ₅ for each constituent Cₓ. In one example, D₁ and D₂ may be negligible compared to D₃, indicating that the intensity information at wavelength λ₃ is primarily influenced by constituent C3. Accordingly, the contribution of the competing constituent C3 can be determined using the intensity at λ₃. This, in turn, allows the contribution from the competing constituent C3 to be subtracted from the intensity measurements at the other wavelengths, such as λ₁, λ₂, λ₄, and λ₅. In some examples, by fitting the intensity changes at the detected wavelengths across a sequence of spectra to the corresponding ratiometric relationships, the computing system can determine the respective contributions of C1 and C2 to the observed spectral changes. In this way, the computing system may determine the concentration of C1 and/or C2.

It will be appreciated that the foregoing example is a simplified illustrative example, and that in practice, a larger number of wavelengths and coefficients may be used, but that the principles discussed above are still applicable. For example, to obtain the optical signatures, over 20 wavelengths may be analyzed. Then, after the optical signatures have been determined, a respective subset of most-relevant wavelengths for a respective constituent may be determined, such that a detection system for determining the constituent may only need to obtain intensity information for a subset of the wavelengths (such as looking at intensities at 7 wavelengths). The detection system thus includes a corresponding set of emitter(s) and detector(s) for obtaining the relevant intensity information at the subset of wavelengths, and may store (or receive) coefficient/ratiometric information for those wavelengths which allows the detection system to determine constituent concentration information for the constituent(s) of interest.

While embodiments of the invention have been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. For example, the various embodiments of the kinematic, control, electrical, mounting, and user interface subsystems can be used interchangeably without departing from the scope of the invention. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A system for dynamically monitoring constituent concentration changes during dialysis, comprising:
a fluid chamber configured to allow a fluid to flow through, wherein the fluid comprises a plurality of constituents, and wherein the fluid is extracorporeal blood or dialysate effluent;
one or more optical sources configured to emit light through the fluid, the light comprising a plurality of wavelengths corresponding to one or more target constituents and one or more competing constituents in the fluid;
one or more optical detectors configured to receive light that has passed through the fluid, the received light corresponding to the plurality of wavelengths; and
one or more processors configured to:
obtain, based on the light received by the one or more optical detectors, intensity information of the light at the plurality of wavelengths; and
determine, in real-time, based on the intensity information of the light at the plurality of wavelengths, a change of a concentration of at least one target constituent of the one or more target constituents in the fluid using a plurality of coefficients based on at least one optical signature corresponding to the at least one target constituent, wherein the plurality of coefficients provide for isolating the at least one target constituent from competing constituents in the fluid, and wherein each of the plurality of coefficients corresponds to a respective intensity information for a respective wavelength.

2. The system according to claim 1, wherein the one or more processors are further configured to perform a response operation based on the determined concentration change of the at least one target constituent, wherein the responsive operation includes adjustment of a treatment parameter or adjustment of a dialysate mixture.

3. The system according to claim 1, wherein the plurality of wavelengths include at least three wavelengths.

4. The system according to claim 1, wherein the one or more processors are further configured to:
obtain optical signatures corresponding to the one or more target constituents and one or more competing constituents, each optical signature indicating a set of wavelengths and relationships between the set of wavelengths corresponding to a respective constituent among the one or more target constituents and one or more competing constituents; and
determine, for a target constituent or a competing constituent among the one or more target constituents and one or more competing constituents, a set of ratiometric coefficients corresponding to the plurality of wavelengths based on the corresponding optical signature;
wherein determining the change of the concentration of the at least one target constituent is based on at least one set of ratiometric coefficients for the at least one target constituent, wherein the at least one set of ratiometric coefficients for the at least one target constituent is comprised in the plurality of coefficients.

5. The system according to claim 4, wherein the one or more processors are further configured to:
determine, based on at least one set of ratiometric coefficients for the at least one competing constituent, a contribution from the at least one competing constituent to the intensity of light, wherein the at least one set of ratiometric coefficients for the at least one competing constituent is comprised in the plurality of coefficients; and
determine a contribution from the at least one targeting constituent to the intensity of light by subtracting the contribution from the at least one competing constituent.

6. The system according to claim 1, wherein the one or more processors are further configured to:
determine one or more wavelengths of the plurality of wavelengths corresponding to a first target constituent of the one or more target constituents; and
determine, based on intensity of light at the one or more wavelengths of the plurality of wavelengths, a change of a concentration of the first target constituent.

7. The system according to claim 1, wherein the one or more optical sources comprise a first optical source and a second optical source, wherein the first optical source is configured to emit light at a first set of wavelengths and the second optical sources is configured to emit light at as a second set of wavelengths, and wherein the first and second sets of wavelengths comprise different wavelengths.

8. The system according to claim 7, wherein the first set of wavelengths comprises one or more wavelengths within the ultraviolet or visible spectrum range, and wherein the second set of wavelengths comprises one or more wavelengths within the visible or infrared spectrum range.

9. The system according to claim 1, wherein the one or more optical detectors comprise at least one miniature solid-state spectrophotometer.

10. The system according to claim 1, wherein the one or more optical sources and the one or more optical detectors are configured to continuously detect light from the fluid.

11. The system according to claim 1, wherein the one or more target constituents and the one or more competing constituents in the fluid are instructed by user input.

12. The system according to claim 1, wherein the one or more target constituents comprise at least one of:
calcium;
potassium;
sodium;
chlorine;
bromine;
bicarbonate;
magnesium;
phosphate;
lactate;
acetate;
creatinine;
glucose;
urea; or
hydrogen peroxide.

13. The system according to claim 1, wherein a target constituent of the one or more target constituents is potassium.

14. The system according to claim 1, wherein a target constituent of the one or more target constituents is calcium.

15. The system according to claim 1, wherein a target constituent of the one or more target constituents is bicarbonate.

16. A method for dynamically monitoring constituent concentration changes during dialysis, comprising:
emitting, by one or more optical sources, light through a fluid that flows through a fluid chamber, the light comprising a plurality of wavelengths corresponding to one or more target constituents and one or more competing constituents in the fluid, wherein the fluid comprises a plurality of constituents, and wherein the fluid is extracorporeal blood or dialysate effluent;
receiving, via one or more optical detectors, light that has passed through the fluid, the received light corresponding to the plurality of wavelengths;
obtaining, by one or more processors, based on the light received by the one or more optical detectors, intensity information of the light at the plurality of wavelengths; and
determining, by the one or more processors, in real-time, based on the intensity information of the light at the plurality of wavelengths, a change of a concentration of at least one target constituent of the one or more target constituents in the fluid using a plurality of coefficients based on at least one optical signature corresponding to the at least one target constituent, wherein the plurality of coefficients provide for isolating the at least one target constituent from competing constituents in the fluid, and wherein each of the plurality of coefficients corresponds to a respective intensity information for a respective wavelength.

17. The method according to claim 16, wherein the plurality of coefficients comprise at least one set of ratiometric coefficients corresponding to at least one target constituent of the one or more target constituents and at least one set of ratiometric coefficients corresponding to at least one competing constituent of the one or more competing constituents, the method further comprising:
determining, based on the at least one set of ratiometric coefficients corresponding to the at least one competing constituent of the one or more competing constituents, a contribution from the at least one competing constituent to the intensity of light; and
determine a contribution from at least one targeting constituent of the one or more targeting constituents to the intensity of light by subtracting the contribution from the at least one competing constituent.

18. The method according to claim 16, wherein a target constituent of the one or more target constituents is potassium.

19. A non-transitory computer-readable medium, having computer-executable instructions stored thereon for dynamically monitoring constituent concentration changes during dialysis, wherein the computer-executable instructions, when executed, facilitate performance of the following:
emitting, by one or more optical sources, light through a fluid that flows through a fluid chamber, the light comprising a plurality of wavelengths corresponding to one or more target constituents and one or more competing constituents in the fluid, wherein the fluid comprises a plurality of constituents, and wherein the fluid is extracorporeal blood or dialysate effluent;
receiving, via one or more optical detectors, light that has passed through the fluid, the received light corresponding to the plurality of wavelengths;
obtaining, by one or more processors, based on the light received by the one or more optical detectors, intensity information of the light at the plurality of wavelengths; and
determining, by the one or more processors, in real-time, based on the intensity information of the light at the plurality of wavelengths, a change of a concentration of at least one target constituent of the one or more target constituents in the fluid using a plurality of coefficients based on at least one optical signature corresponding to the at least one target constituent, wherein the plurality of coefficients provide for isolating the at least one target constituent from competing constituents in the fluid, and wherein each of the plurality of coefficients corresponds to a respective intensity information for a respective wavelength.

20. The non-transitory computer-readable medium according to claim 19, wherein the plurality of coefficients comprise at least one set of ratiometric coefficients corresponding to at least one target constituent of the one or more target constituents and at least one set of ratiometric coefficients corresponding to at least one competing constituent of the one or more competing constituents, the computer-executable instructions, when executed, further facilitate performance of the following:
determining, based on the at least one set of ratiometric coefficients corresponding to the at least one competing constituent of the one or more competing constituents, a contribution from the at least one competing constituent to the intensity of light; and
determine a contribution from at least one targeting constituent of the one or more targeting constituents to the intensity of light by subtracting the contribution from the at least one competing constituent.
